(19) United States
(12) Patent Application Publication
(10) Pub. No.: US 2025/0279179 A1
(43) Pub. Date: Sep. 4, 2025

(54) METHODS, DEVICES, AND COMPOSITIONS TO MEET NUTRITIONAL NEEDS IN AN ENVIRONMENTALLY SUSTAINABLE MANNER

(71) Applicant: SOCIETE DES PRODUITS NESTLE S.A., Vevey (CH)

(72) Inventors: DANTONG WANG, Prilly (CH); FABIO MAINARDI, Lausanne (CH); DIANE ZIMMERMANN, Feigeres (FR); NAMY DANIELA ESPINOZA ORIAS, Lausanne 25 (CH); EMMA FRANCES JACQUIER, Chatel-St-Denis (CH); IAROSLAVA SEMENOVA, Lausanne (CH); IVAN MONTOLIU ROURA, Lausanne 26 (CH)

(21) Appl. No.: 18/548,608

(22) PCT Filed: Mar. 4, 2022
(86) PCT No.: PCT/EP2022/055518

§ 371 Date: Sep. 1, 2023

Related U.S. Application Data
(60) Provisional application No. 63/157,322, filed on Mar. 5, 2021.

(51) Int. Cl. G16H 20/60; G06Q 30/0601

(52) U.S. Cl. CPC G16H 20/60; G06Q 30/0633

(57) ABSTRACT

A tailored dietary product recommendation tool, more specifically, a tool for providing optimized nutrition recommendations and products is provided in an environmentally sustainable manner tailored to a dietary pattern of a user. The tool can be used for preventing and/or reducing a risk of a nutrient inadequacy, achieving a nutrient adequacy, meeting a nutrition need, and/or minimizing a nutrient gap in a subject in need thereof in an environmentally sustainable manner. For example, a nutrition need of the user can be assessed. Then, potential environmental impacts can be estimated for combinations of existing food products that meet the nutrient need of the user. The combination with the lowest environmental impact can be recommended to the user, who can then place an order accordingly. The producer can pre-pack existing products according to the order and ship them to the user.

### CROSS REFERENCE TO RELATED APPLICATIONS

The present application is a National Stage of International Application No. PCT/EP2022/055518, filed on Mar. 4, 2022, which claims priority to U.S. Provisional Patent Application No. 63/157,322, filed Mar. 5, 2021, the entire contents of which are being incorporated herein by reference.

### BACKGROUND

The present disclosure generally relates to providing nutrients to a subject. More specifically, it relates to a method, device, and composition for meeting the subject's nutritional needs in an environmentally sustainable manner.

People's dietary habits are different, and different dietary patterns result in different nutritional needs in the population. Dietary patterns differ not only in terms of food intake characteristics but also nutrient adequacy between people. Many food products, particularly those consumed regularly, such as milk and breakfast cereals, have different nutrition compositions, and each focus on limited nutrients. Such mass-market products are not necessarily adapted to specific consumer needs. Consumers may need to buy multiple products or change products frequently to meet their nutritional needs, which can be costly, time-consuming and can create a large volume of waste from packaging. For mass-market producers, it can be challenging to meet the tailored needs of consumers due to manufacturing and packaging format constraints.

Currently, if consumers would like to purchase products based on their nutritional needs, first they need to assess their dietary intake quality and identify nutrition gaps, then purchase products one by one using information on product label. There are apps or websites available to evaluate people's dietary intake by answering a long list of questions and providing recommendations. However, the reliability of the evidence used by such tools is uncertain; the nutrition recommendations still need to be translated into products; and the consumers still need to purchase the products one-by-one, and no product can be pre-packed since there is no production combination recommendation.

However, it is difficult for consumers to understand their nutritional needs. Further, it is challenging for consumers to identify which product provides suitable nutrition. The consumers need to compare their nutritional needs with products' nutrition information. Moreover, consumers often need to purchase multiple products packaged individually to meet their nutritional needs, which could potentially have a worse environmental sustainability performance and is potentially more costly.

### SUMMARY

This disclosure can solve the problems discussed above. The present disclosure provides a tool for optimizing the combination of existing products, such as different variants of the same brand, based on consumers' nutritional needs. With this approach, the combination of products can be packed in a more environmentally sustainable manner, for example, with an estimated reduction on the climate change impact by 50-70% when there is a change in the product format and/or packaging material and by 20% when only the size of the package is changed.

In one aspect, provided is a method of preventing and/or reducing a risk of a nutrient inadequacy, achieving a nutrient adequacy, meeting a nutrition need, and/or closing a nutrient gap in a subject in need thereof in an environmentally sustainable manner. The method may include assessing a nutrition need of the subject; providing combinations of existing food products that meet the nutrient need of the subject; estimating potential environmental impacts of the combinations of the existing food products; selecting a combination of the existing products with the lowest environmental impact; placing an order of the combination of the existing products with the lowest environmental impact; and providing a pre-packed combination of the existing products with the lowest environmental impact according to the order.

In one embodiment, the assessing of the nutrition need of the subject may comprise calculating a nutrient gap of the subject.

In one embodiment, the nutrient gap may be calculated as (a daily nutrient intake recommendation−an average nutrient intake in a cluster of subjects)/the daily nutrient intake recommendation. The nutrient intakes within the cluster are more similar to each other than to those in other clusters.

In one embodiment, the assessing of the nutrition need of the subject may comprise using a questionnaire including about 1-10 questions. In one embodiment, the questionnaire includes about 5-6 questions.

In one embodiment, the existing food products each may be selected from the group consisting of a powdered nutritional composition to be reconstituted in milk or water, a powdered nutritional composition stored in a single use capsule or pod to be reconstituted in milk or water, a nutritional formula, a cereal-based product, a drink, a bar, a nutritional supplement, a pre-mix for fortification purposes, a nutraceutical, a yogurt, a dairy-based product, a food sprinkler, a gummy, a meal replacer, a pill, a tablet, and combinations thereof.

In one embodiment, the existing food products each may be in a form selected from the group consisting of a powder, a ready-to-drink (RTD) product, and combinations thereof.

In one embodiment, the existing food products each may be in a package selected from the group consisting of can, sachet, bottle, and combinations thereof.

In one embodiment, the existing food products may be beverages, cereal products or pet foods.

In one embodiment, the combinations of the existing food products each may comprise a combination of proportions of product variants and/or numbers of product units.

In one embodiment, the combinations of the existing food products may provide nutrients comprising at least one of carbohydrates, proteins, fibers, vitamins, fats, prebiotics, probiotics and minerals.

In one embodiment, the estimating of the potential environmental impacts of the combinations of the existing food products may comprise performing a screening life cycle assessment of factors selected from the group consisting of packaging material manufacture, filling at factory, distribution, retail, transport within life cycle stages, the end of life management of used packaging, and combinations thereof. In one embodiment, the factor does not include manufacture of the product per se (e.g. recipe) and consumption of the existing products at home/on the go. The screening life cycle assessment preferably focuses on all life cycle stages that are different for the various products tested.

In one embodiment, the estimating of the potential environmental impacts may comprise estimating an environmental impact factor per serving size(s) delivered and selected from the group consisting of climate change (CC) [kg CO2-eq], freshwater consumption scarcity (FWCS) [m3-eq], abiotic resource depletion (ARD) [kg Sb-eq], land use impacts on biodiversity (LUIB) [PDF*m2*year], and impacts on ecosphere/ecosystems quality (IEEQ) [PDF*m2*year], and combinations thereof.

In one embodiment, at least one of the following steps of the method may be implemented on a computer machine: the assessing of nutrition need of the subject: the providing of the combinations of existing food products that meet the nutrient need of the subject; the estimating of the potential environmental impact value for each of the combinations of the existing food products; the selecting of the combination of the existing food products with a lowest potential environmental impact value; or the placing of the order of the combination of the existing food products with the lowest potential environmental impact.

In another aspect, a computer system may comprise at least one processor; and a data storage device in communication with the at least one processor, wherein the data storage device may comprise instructions that, when executed by the at least one processor, cause the at least one processor to assess a nutrition need of the subject; provide combinations of existing food products that meet the nutrient need of the subject; estimate a potential environmental impact value for each of the combinations of the existing food products; select a combination of the existing food products with a lowest potential environmental impact value; and place an order of the combination of the existing food products with the lowest potential environmental impact.

In yet another aspect, a computer readable medium may comprise instructions which, when executed by a computer, may cause the computer to assess a nutrition need of the subject, provide combinations of existing food products that meet the nutrient need of the subject; estimate a potential environmental impact value for each of the combinations of the existing food products: select a combination of the existing food products with a lowest potential environmental impact value; and place an order of the combination of the existing food products with the lowest potential environmental impact.

An advantage of the present disclosure is a simplified questionnaire which reduces the burden of the user, improves the user experience, and provides direct proposals on how to combine the existing products. This helps both consumers and producers. Consumers can use this tool to assess their nutritional needs by answering only a few questions, such as 5 to 6 questions and receive nutrition recommendation and proposed combinations of existing products that provide optimized nutrition with online order link. Producers can pre-pack products according to consumers' orders and ship them to consumers with reduced environmental impact.

The present invention provides for a method of preventing nutrient inadequacies in a subject. For example, the method according to the present disclosure reduces the risk of nutrient inadequacies by helping to achieving nutrient adequacy to support the nutritional needs of the subject.

### BRIEF DESCRIPTION OF THE DRAWINGS

FIG. 1 schematically represents the system boundaries for the scenarios assessing a screening life cycle assessment. Excluded life cycle stages are underlined (ingredients and consumption).

FIGS. 2 and 3 are representations of the results for climate change impact for Scenarios A to D of Molino product per week of nutrition delivered. The X-axis shows the Scenario type. The Y-axis shows the climate change (CC) [kg CO₂-eq] (100 a, IPCC, 2013).

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all ratios are by weight unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 1 to 10 (including 1 and 10), from 2 to 8, from 3 to 7, from 1 to 9, from 3.6 to 4.6, from 3.5 to 9.9, and so forth. All references to singular characteristics or limitations of the present invention shall include the corresponding plural characteristic or limitation, and vice versa, unless otherwise specified or clearly implied to the contrary by the context in which the reference, is made.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of the “X and/or Y” should be interpreted as “X”, or “Y”, or “X and Y”.

As used herein and in the appended claims. “A and/or B” means “A without B,” or “B without A,” or “both A and B.”

Where used herein, the term “example,” particularly when followed by a listing of terms, is merely exemplary and illustrative and should not be deemed to be exclusive or comprehensive. 30 Any embodiment disclosed herein can be combined with any other embodiment disclosed herein unless explicitly indicated otherwise.

The terms “food,” “food product” and “food composition” mean a composition that is intended for ingestion by an individual, such as a human, and that provides at least one nutrient to the individual. The term “pet food” mean a composition that is intended for ingestion by an animal such as a pet.

The term “order” indicates any message to a manufacturer, producer, factor, distributor, shop, store and/or any other type of product provider that identifies a product or products to be purchased, optionally with a mailing, delivery, or pickup location, and optionally with payment information.

Within the context of the present invention, the term “nutrient” or nutrients” is intended to comprise both macronutrients (for example carbohydrates, proteins or fats) and micronutrients (for example minerals or vitamins) for the human or animal body.

Within the context of the present invention, the terms “ingredient” or “ingredients” indicate an edible substance or mixture of substances which comprise or is essentially consisting of a nutrient for the human or animal body. In one embodiment of the present invention, the terms “ingredient” or “ingredients” indicate an edible substance essentially consisting of a nutrient for the human or animal body.

Within the context of the present invention, the term “ingredient X” or “(nutrient) X” indicates an edible substance or mixture of substances which comprise or is essentially consisting of at least one substance capable of delivering the specified nutrient X to the human or animal body. In one embodiment, the term “ingredient X” or “(nutrient) X” indicates an edible substance essentially consisting of substance capable of delivering the specified nutrient X to the human or animal body.

Within the context of the present invention, the terms “ingredient providing nutrient X” or “ingredients providing nutrient X” indicate an edible substance and/or mixture of substances which comprise or is essentially consisting of at least one substance capable of delivering the specified nutrient X to the human or animal body.

Within the context of the present invention, the terms “ingredient providing nutrient X in amount Y” or “ingredients providing nutrient X in amount Y” indicate an edible substance and/or mixture of substances which comprise or is essentially consisting of at least one substance capable of delivering the specified nutrient X to the human or animal body in the specified amount Y.

The expressions “fiber” or “fibers” or “dietary fiber” or “dietary fibers” within the context of the present invention indicate the indigestible portion, in small intestine, of food derived from plants which comprises two main components: soluble fiber, which dissolves in water, and insoluble fiber. Mixtures of fibers are comprised within the scope of the terms above mentioned. Soluble fiber is readily fermented in the colon into gases and physiologically active byproducts, and can be prebiotic and viscous. Insoluble fiber does not dissolve in water, is metabolically inert and provides bulking, or it can be prebiotic and metabolically ferment in the large intestine. Chemically, dietary fiber consists of (1) non-starch polysaccharides (NSP) such as arabinoxylans, cellulose, beta-glucans and many other plant components such as inulin, pectins or gums: (2) resistant oligosaccharides such as resistant dextrins; (3) resistant starch and (4) associated substances from plants such as lignin or chitins. Non limiting examples of dietary fibers are: prebiotic fibers such as Fructo oligosaccharides (FOS), inulin, galacto-oligosaccharides (GOS), fruit fiber, legume fiber, vegetable fiber, cereal fiber, resistant starch such as high amylose corn starch. As fibers are not digestible, they do not contain available carbohydrates.

The total fiber content of the food composition is provided by the sum of amount of fiber naturally present in all the ingredients used in the recipe (for example from whole grain cereal flour, from fruit or vegetables).

As it will be apparent to the skilled person, grain products, bread, breakfast cereals, potatoes, vegetables, legumes and fruits according to the present invention may bring certain amount of fibers to the food composition of the present invention.

Within the context of the present invention, the term “nutrient inadequacy” or “dietary inadequacy” indicates that the total daily dietary intake of a nutrient in a certain subject is below the EFSA's daily intake recommendation for the gender and age group the subject belongs to and/or below well-established nutritional requirement for that group.

Within the context of the present invention, the expression “prevent nutrient inadequacy” should be understood to include prevention of nutrient inadequacies as well as reduction of the risk of nutrient inadequacies in a subject.

In one aspect of the present disclosure, a method is provided for recommending an optimized combination of existing products in an environment-friendly manner to meet a user's nutrition need. This method can be used by a consumer and/or a factory. The method may include the following parts, for example, Cluster Analysis for analysing a dietary pattern of the subject; a Decision Tree process for defining the dietary pattern of the subject; and an optimizer for providing recommendations to the subject.

Cluster Analysis may be used to classify dietary patterns by pre-analyzing the dietary intake data of the population. Nutrition gaps of each dietary pattern can be identified by comparing the intakes to the recommendations.

Cluster Analysis is the study of how to partition a set of items into non-overlapping groups, or clusters, such that items within a cluster are more similar to each other than to items in other clusters. The algorithm K-means can be used. The number of clusters may be chosen using the gap statistic approach, basically taking into consideration the amount of variation explained by the solution, the size and interpretability of each cluster, and the stability of the solution, which was evaluated using linear discriminant analysis.

A Decision Tree method may be applied to define the dietary pattern of a consumer with a small number of questions. For example, within 6 questions, a dietary pattern of the consumer can be defined with 90% accuracy. The Decision Tree may not be the same for all geographies and may depend on the local dietary habits. It can be adjusted if dietary intake data is available.

The Decision Tree can be developed according to statistical methodologies. For example, a classification tree can be a tree in which each internal node corresponds to a partition of the data according to a cut-off for a single feature (in this case, the intake of a food group in the given population). Navigating the tree from the top to the bottom may lead to a value of the response variable, for example, the index of a cluster. The choice of the cut-offs and their order can be done algorithmically, as the result of fitting the tree model to the data. For example, the R implementation in the package rpart can be used.

For example, first, the dataset can be randomly split into a training set and a test set (80%¬20% split). Then, the tree can be developed on the training data; a process called pruning can then be performed in order to reduce the complexity of the tree and therefore avoiding the risk of overfitting. Overfitting corresponds to selecting an overly complex model, with more parameters that can be justified by the data, and leads to poor predictive performance. The pruning process not only can reduce the risk of overfitting, but also can reduce the depth of the tree, e.g., the number of questions. Last, the tree can be applied to the test data, where the predictive performance may be evaluated, by looking at the sensitivity and specificity for each cluster. The random train/test split can be repeated several times in order to average out the variability induced by the split and to minimize the risk of picking some features by chance.

An optimizer may be used to propose combinations of existing products that provide the most nutrients in need and the pre-pack information.

The method according to the present disclosure may be used for preventing and/or reducing a risk of a nutrient inadequacy, achieving a nutrient adequacy, meeting a nutrition need, and/or minimizing a nutrient gap in a subject in need thereof in an environmentally sustainable manner.

The method may include assessing a nutrition need of the subject: providing combinations of existing food products that meet the nutrient need of the subject: estimating potential environmental impacts of the combinations of the existing food products; selecting a combination of the existing products with the lowest environmental impact: placing an order of the combination of the existing products with the lowest environmental impact; and providing a pre-packed combination of the existing products with the lowest environmental impact according to the order.

In one embodiment, the assessing of the nutrition need of the subject may comprise calculating a nutrient gap of the subject.

In one embodiment, the nutrient gap may be calculated as (a daily nutrient intake recommendation−an average nutrient intake in a cluster of subjects)/the daily nutrient intake recommendation. The nutrient intakes within the cluster are more similar to each other than to those in other clusters.

In one embodiment, the assessing of the nutrition need of the subject may comprise using a questionnaire including about 1-10 questions. In one embodiment, the questionnaire includes about 5-6 questions.

In one embodiment, the existing food products each may be selected from the group consisting of a powdered nutritional composition to be reconstituted in milk or water, a powdered nutritional composition stored in a single use capsule or pod to be reconstituted in milk or water, a nutritional formula, a cereal-based product, a drink, a bar, a nutritional supplement, a pre-mix for fortification purposes, a nutraceutical, a yogurt, a dairy-based product, a food sprinkler, a gummy, a meal replacer, a pill, a tablet, and combinations thereof.

In one embodiment, the existing food products each may be in a form selected from the group consisting of a powder, a ready-to-drink (RTD) product, and combinations thereof.

In one embodiment, the existing food products each may be in a package selected from the group consisting of can, sachet, bottle, and combinations thereof.

In one embodiment, the combinations of the existing food products each may comprise a combination of proportions of product variants and/or numbers of product units.

In one embodiment, the combinations of the existing food products may provide nutrients comprising at least one of carbohydrates, proteins, fibers, vitamins, fats, prebiotics, probiotics, vitamins, and minerals.

In one embodiment, the estimating of the potential environmental impacts of the combinations of the existing food products may comprise performing a screening life cycle assessment of a factors selected from the group consisting of packaging material manufacture, filling at factory, distribution, retail, transport within life cycle stages, the end of life management of used packaging, and combinations thereof. In one embodiment, the factor does not include manufacture and consumption of the existing products.

In one embodiment, the estimating of the potential environmental impacts may comprise estimating an environmental impact factor per serving size(s) delivered selected from the group consisting of climate change (CC) [kg CO₂-eq], freshwater consumption scarcity (FWCS) [m³-eq], abiotic resource depletion (ARD) [kg Sb-eq], land use impacts on biodiversity (LUIB) [PDF*m²*year], and impacts on ecosphere/ecosystems quality (IEEQ) [PDF*m²*year], and combinations thereof.

In one embodiment, the placing of the order of the combination of the existing products may comprise sending a provider of the existing products a message identifying the combination of the existing products that the subject intends to purchase, optionally with a mailing, delivery, or pickup location, and optionally with payment information.

In one embodiment, providing a pre-packed article in reply to the order may include shipping of the pre-packed article from a first location to a second location. For example, the first location may be a warehouse, a factory, a store, or any other location where the ordered combination of the existing products are available, stored, or pre-packed. The second location may be a home address of the subject or any mailing, delivery, or pickup address the subject provided in the order.

In one embodiment, the methods described herein may be implemented on a computer machine.

In another aspect of the present disclosure, a data processing device may comprise means for carrying out the computer implemented methods described herein.

In another aspect, a computer readable medium may comprise instructions which, when executed by a data processing device, such as a computer, cause the data processing device to carry out the methods described herein.

In another aspect, the present disclosure provides an optimized food composition that meets a subject's nutritional needs and is environmentally sustainable. The optimized food composition can include a combination of existing food products.

The compositions described according to the present invention, including the many embodiments described herein, can comprise, consist of, or consist essentially of the essential elements and limitations of the invention described herein, as well as any additional or optional ingredients, components, or limitations described herein.

The food compositions according to the present disclosure can provide nutrients comprising carbohydrates, total protein, dietary fiber, calcium, iron, magnesium, potassium, zinc, and vitamin C, among others.

The food compositions according to the present disclosure may comprise ingredients which are sources of nutrients such as proteins, fats, carbohydrates, prebiotics, probiotics, vitamins and/or minerals.

The following are non-limiting examples of nutrients that may be provided by the food composition according to the present disclosure.

### Carbohydrates

In one embodiment of the present invention, a food composition is provided which comprises carbohydrate. Carbohydrates may be incorporated in the composition as such and/or via any source comprising carbohydrates. For example, ingredients may be selected in the group consisting of monosaccharides, disaccharides, oligosaccharides, polysaccharides or a mixture thereof.

Different ingredients may provide different amounts of carbohydrates in the composition according to the present invention, depending on the nature and amount of the ingredient used. In one embodiment, the food composition of the present invention delivers the total daily amount of carbohydrates considered necessary to address the nutrient gap.

### Protein

In one embodiment of the present invention, a food composition is provided which comprises protein. Protein may be incorporated in the composition of the invention as such or and/or via any source comprising protein. For example, ingredients may be selected in the group consisting of: animal protein, dairy protein, plant protein or a mixture thereof.

Different ingredients may provide different amounts of protein in the composition according to the present invention, depending on the nature and amount of the ingredient used. In one embodiment, the food composition delivers the total daily amount of protein considered necessary to address the nutrient gap.

### Calcium

In one embodiment of the present invention, a food composition is provided which comprises calcium. Calcium may be incorporated in the composition of the invention as such or in the form of a physiologically acceptable salt and/or via any source comprising calcium. For example, ingredients may be selected in the group consisting of: calcium sulfate, calcium citrate, calcium chloride, calcium fumarate, calcium gluconate or a mixture thereof.

Different ingredients may provide different amounts of calcium in the composition according to the present invention, depending on the nature and amount of the ingredient used. In one embodiment, the food composition delivers the total daily amount of calcium considered necessary to address the nutrient gap.

### Iron

In one embodiment of the present invention, a food composition is provided which comprises iron. Iron may be incorporated in the composition of the invention as such or in the form of a physiologically acceptable salt and/or via any source comprising iron. For example, ingredients may be selected in the group consisting of: iron sulfate, iron citrate, iron choline citrate, iron ammonium citrate, iron chloride, iron fumarate, iron gluconate, iron pyrophosphate or a mixture thereof.

Different ingredients may provide different amounts of iron in the composition according to the present invention, depending on the nature and amount of the ingredient used. In one embodiment, the food composition of the present invention delivers the total daily amount of iron considered necessary to address the nutrient gap.

### Magnesium

In one embodiment of the present invention, a food composition is provided which comprises magnesium. Magnesium may be incorporated in the composition of the invention as such or in the form of a physiologically acceptable derivative and/or via any source comprising magnesium. For example, ingredients may be selected in the group consisting of: magnesium phosphate, magnesium sulfate, magnesium citrate, magnesium chloride, magnesium aspartate, magnesium bicarbonate, magnesium gluconate or a mixture thereof.

Different ingredients may provide different amounts of magnesium in the composition according to the present invention, depending on the nature and amount of the ingredient used. In one embodiment, the food composition of the present invention delivers the total daily amount of magnesium considered necessary to address the nutrient gap.

### Fibers

In one embodiment of the present invention, a food composition is provided which comprises fibers. Fibers may be incorporated in the composition of the invention as such and/or via any source comprising fibers. For example, ingredients may be selected in the group consisting of: fruit, vegetable, legume, cereal and cruciferous vegetable.

In one embodiment, dietary fibers are selected in the group consisting of: resistant oligosaccharides (such as resistant dextrin, polydextrose or FOS), NSP (for example pectin, inulin, partially hydrolyzed guar gum (PHGG), acacia gum), resistant starches and mixtures thereof. In one embodiment, the ingredients providing fibers may be capable of providing fibers of natural or synthetic origin. In one embodiment, fibers of synthetic origin are for example FOS from sucrose.

Different ingredients may provide different amounts of fibers in the composition according to the present invention, depending on the nature and amount of the ingredient used. In one embodiment, the food composition of the present invention delivers the total daily amount of fibers considered necessary to address the nutrient gap.

### Potassium

In one embodiment of the present invention, a food composition is provided which comprises potassium. Potassium may be incorporated in the composition of the invention as such or in the form of a physiologically acceptable salt and/or via any source comprising potassium. For example, ingredients may be selected in the group consisting of: potassium phosphate, potassium sulfate, potassium citrate, potassium chloride, potassium aspartate, potassium bicarbonate, potassium gluconate or a mixture thereof.

Different ingredients may provide different amounts of potassium in the composition according to the present invention, depending on the nature and amount of the ingredient used. In one embodiment, the food composition of the present invention delivers the total daily amount of potassium considered necessary to address the nutrient gap.

### Zinc

In one embodiment of the present invention, a food composition is provided which comprises zinc. Zinc may be incorporated in the composition of the invention as such or in the form of a physiologically acceptable salt and/or via any source comprising zinc. For example, ingredients may be selected in the group consisting of: zinc acetate, zinc chloride, zinc citrate, zinc gluconate, zinc lactate, zinc oxide, zinc sulphate, zinc carbonate and mixtures thereof.

Different ingredients may provide different amounts of zinc in the composition according to the present invention, depending on the nature and amount of the ingredient used. In one embodiment, the food composition of the present invention delivers the total daily amount of zinc considered necessary to address the nutrient gap.

### Vitamin C

In one embodiment of the present invention, a food composition is provided which comprises vitamin C. Vitamin C may be incorporated in the composition of the invention as such or in the form of a physiologically acceptable salt and/or via any source comprising vitamin C. For example, ingredients may be selected in the group consisting of: broccoli. Brussels sprouts, and cauliflower, green and red peppers, leafy green vegetables, ascorbic acid, sodium ascorbate and mixtures thereof.

Different ingredients may provide different amounts of vitamin C in the composition according to the present invention, depending on the nature and amount of the ingredient used. In one embodiment, the food composition of the present invention delivers the total daily amount of vitamin C considered necessary to address the nutrient gap.

It is to be understood that the compositions according to the present disclosure may provide other nutrients not described above but that are needed by the subject.

The food compositions according to the present disclosure may be selected from the group consisting of a powdered nutritional composition to be reconstituted in milk or water, a powdered nutritional composition stored in a single use capsule or pod to be reconstituted in milk or water, a nutritional formula, a cereal-based product, a drink, a bar, a nutritional supplement, a pre-mix (for example of minerals and vitamins) for fortification purposes, a nutraceutical, a yogurt, a dairy-based product, a food sprinkler, a gummy, a meal replacer, a pill, a tablet, and combinations thereof.

For example, in one embodiment, the food composition may be a dairy-based product, for example a liquid dairy-based product or a powdered dairy-based product to be reconstituted in milk or water. In another embodiment, the food composition may be a nutritional supplement, for example in the form of a tablet or a gummy. The supplement may provide selected nutrients while not representing a significant portion of the overall nutritional needs of the subject and/or does not represent more than 0.1%, 1%, 5%, 10%, or 20% of the daily energy need of the subject. In another embodiment, the food composition may be a beverage, a cereal product or a pet food, for example.

The principles and operation of the methods, devices, and compositions according to the present disclosure may be better understood with reference to the Examples and the accompanying description, it being understood that figures and tables in the experimental section are given for illustrative purposes only and are not meant to be limiting.

### EXAMPLES

The following non-limiting examples present scientific data developing and supporting the concept of the methods, devices, and compositions of the present disclosure. In the Examples. Molico in Brazil was used for illustration.

First, the dietary intake data in Brazil was analysed by Cluster Analysis. Four dietary patterns were identified, and the nutrition inadequacy of each dietary pattern was assessed. Second, Decision Tree approach was applied to reduce the number of questions that can distinguish different dietary patterns. Third, an Optimizer was used to calculate the optimal combination of existing products based on the product nutrition composition. The proportion of each product was given in order to obtain the optimal nutrition intake from the existing products. Fourth, the environmental impact of three packaging scenarios was measured.

### Example 1: Dietary Intake Cluster and Nutrient Gaps Assessment

The approach used to derive the dietary patterns was based on cluster analysis, specifically on a version of the algorithm K-means: sparse and robust K-means. The analyses were performed for 3 to 5 clusters. Four clusters were found in this example.

For each cluster, the nutrient gaps were estimated based on % of daily recommendations. The optimizer aims at minimizing these nutrient gaps by adding a combination of (any) products. The combination can be intended as proportions of each product variant or as the number of product units for a given time period (e.g., a week). Additional constraints can be imposed, for example, to limit the total sodium or energy intake or to require a minimum amount of fiber. This minimization problem can be formulated as a linear programming (LP) problem and solved accordingly. Efficient algorithms to solve LP are available in standard computing languages, such as R (package 1 psolve) or Python (package or Tools). A Shiny interactive application can be used to simulate different choices of parameters.

TABLE 1
Dietary intake clusters and nutrient gap for each cluster. Calculated as (daily
recommendation − average nutrient intake in cluster)/daily recommendation

| | | Total | | | | Dietary | | | |
|---|---|---|---|---|---|---|---|---|---|
| Cluster | Carbohydrate | Protein | Calcium | Iron | Magnesium | Fiber | Potassium | Zinc | Vitamin C |
| 1 | 58% | −54% | 46% | 50% | 36% | 36% | 18% | 2% | 36% |
| 2 | 58% | −63% | 57% | 43% | 32% | 36% | 21% | −19% | 96% |
| 3 | 64% | −34% | 62% | 62% | 54% | 63% | 45% | 1% | 96% |
| 4 | 61% | −40% | 40% | 53% | 44% | 55% | 33% | −14% | 92% |

In Table 1, the units for each dietary intake are indicated in parentheses: Carbohydrate (g); Total Protein (g); Calcium (mg); Iron (mg); Magnesium (mg); Dietary fiber (g); Potassium (mg); Zinc (mg); Vitamin C (mg).

The nutrient intake distribution of each cluster was calculated. Table 2 below reports the nutrients' mean daily intake (Mean) and standard deviation (SD) as well as the nutrients' median daily intake (Median) and minimum and maximum range (Min, Max) in each cluster.

TABLE 2
Nutrient intake distribution of each dietary intake cluster

| | 1 | | 2 | | 3 | | 4 | |
|---|---|---|---|---|---|---|---|---|
| | (N = 174) | | (N = 140) | | (N = 45) | | (N = 31) | |
| Energy | | | | | | | | |
| Mean (SD) | 1520 | (634) | 2120 | (727) | 1610 | (627) | 1620 | (734) |
| Median [Min, Max] | 1350 | [709, 3860] | 1990 | [832, 3990] | 1710 | [816, 3490] | 1390 | [818, 3990] |
| AnimalProtein | | | | | | | | |
| Mean (SD) | 41.5 | (37.9) | 53.2 | (40.7) | 32.7 | (22.0) | 47.7 | (35.0) |
| Median [Min, Max] | 30.7 | [0, 232] | 43.1 | [0, 227] | 28.1 | [6.35, 101] | 36.8 | [0, 161] |
| DairyProtein | | | | | | | | |
| Mean (SD) | 7.55 | (9.11) | 4.78 | (6.48) | 8.50 | (9.92) | 5.14 | (5.06) |
| Median [Min, Max] | 5.40 | [0, 69.7] | 3.19 | [0, 40.3] | 6.48 | [0, 45.0] | 4.78 | [0, 15.9] |
| PlantProtein | | | | | | | | |
| Mean (SD) | 18.9 | (9.93) | 32.6 | (11.6) | 27.5 | (12.9) | 26.2 | (12.0) |
| Median [Min, Max] | 17.3 | [1.41, 67.5] | 30.4 | [13.9, 75.0] | 25.9 | [6.83, 67.1] | 23.3 | [11.0, 55.5] |
| SaturatedFat | | | | | | | | |
| Mean (SD) | 11.3 | (7.39) | 6.54 | (3.80) | 11.0 | (4.89) | 8.53 | (3.43) |
| Median [Min, Max] | 9.89 | [1.00, 62.2] | 8.18 | [2.15, 21.7] | 10.5 | [4.14, 26.5] | 8.29 | [1.53, 17.1] |
| MUFA | | | | | | | | |
| Mean (SD) | 10.6 | (4.92) | 9.52 | (3.69) | 11.3 | (3.41) | 10.6 | (4.35) |
| Median [Min, Max] | 10.2 | [0.928, 32.0] | 8.97 | [3.11, 22.8] | 11.4 | [5.23, 20.2] | 10.5 | [1.40, 21.7] |
| DietaryFiber | | | | | | | | |
| Mean (SD) | 10.0 | (6.03) | 16.8 | (7.33) | 14.0 | (6.35) | 19.5 | (11.0) |
| Median [Min, Max] | 8.66 | [0.720, 42.4] | 14.9 | [6.30, 50.0] | 13.4 | [3.21, 29.1] | 17.5 | [8.43, 60.9] |
| Sodium | | | | | | | | |
| Mean (SD) | 2520 | (1310) | 3880 | (1510) | 3280 | (1360) | 3080 | (1290) |
| Median [Min, Max] | 2260 | [137, 10000] | 3450 | [1630, 9630] | 2930 | [709, 7150] | 2880 | [699, 6170] |
| Potassium | | | | | | | | |
| Mean (SD) | 1.63 | (0.798) | 2.28 | (1.16) | 1.95 | (0.793) | 2.61 | (1.19) |
| Median [Min, Max] | 1.50 | [0.327, 4.81] | 1.96 | [0.819, 8.29] | 1.93 | [0.487, 3.76] | 2.35 | [1.36, 7.87] |

In Table 2, the units for each dietary intake are indicated in parentheses: Energy (kcal): Animal Protein (g); Dairy Protein (g); Plant Protein (g); Saturated Fat (g); MUFA (g); Dietary Fiber (g); Sodium (mg); Potassium (g).

As shown in Tables 1-2, four dietary intake clusters were identified, and each cluster with different nutrition gaps. By applying the Optimizer, four product combination scenarios (one for each dietary intake cluster) were proposed (Table 3).

### Example 2: Reduced Number of Questions to Define the Dietary Pattern

The Decision Tree approach was utilized to reduce the number of questions that can distinguish different dietary patterns.

### Example 3: Proposed Combination Scenarios

The Optimizer was applied to propose combination of products. The optimizer aims at minimizing these nutrient gaps, by adding a combination of (any) products.

The combination can be intended as proportions of each product variant, or as the number of product units for a given time period (e.g. a week). Additional constraints can be imposed, for example to limit the total sodium or energy intake, or to ask for a minimum amount of fiber. This minimization problem can be formulated as a linear programming (LP) problem and solved accordingly. Efficient algorithms to solve LP are available in standard computing languages, such as R (package lpsolve) or Python (package or Tools). A Shiny interactive application was developed in the prototyping phase in order to simulate different choices of parameters.

By applying the Optimizer, 4 product combination scenarios (one for each dietary intake cluster) were proposed as shown below in Table 3.

TABLE 3
Optimized combinations of MOLICO product variants

| Brazil | | | | | |
|---|---|---|---|---|---|
| Dietary | Molico Product Variants | | | | |
| cluster/combination | | Plant | | Lactose | |
| scenario | Skim | based | Collagen | free | Fibers |
| 1/A | 3 | 2 | 2 | 0 | 0 |
| 2/B | 4 | 1 | 0 | 0 | 2 |
| 3/C | 2 | 4 | 0 | 0 | 1 |
| 4/D | 0 | 1 | 0 | 4 | 2 |

### Example 4: Environmental Impact Assessment

A screening life cycle assessment was performed to understand the potential environmental impact of delivering the optimized nutritional supplements to consumers when using three types of packaging: cans and sachets for a product in powder format and bottles for a product in a “Ready to Drink (RTD)” format.

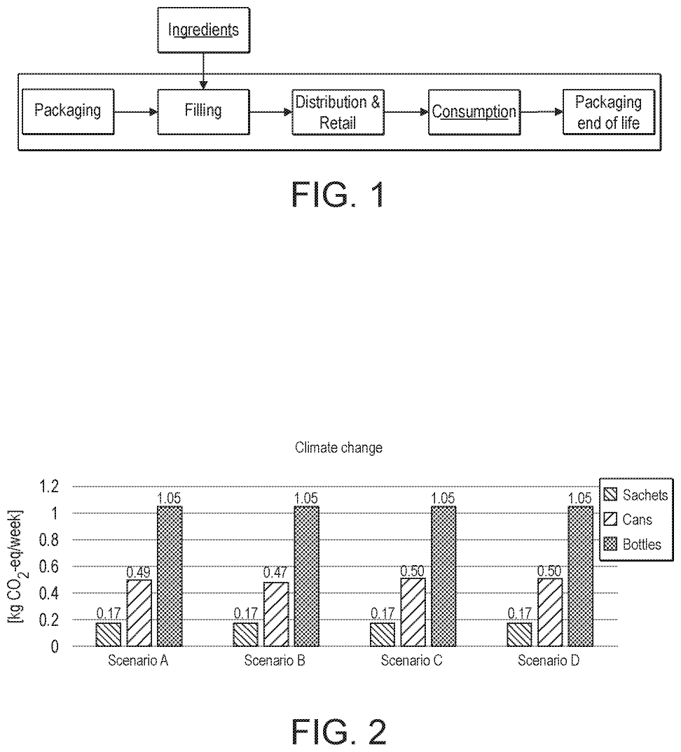

A “serving size of 20 g of powder product at consumers' hands” was chosen as a unit of analysis for all scenarios. The RTD format delivers the reconstituted beverage conveniently, whereas the powder format requires reconstitution at the point of consumption. The boundaries of the cradle to gate assessments are shown in FIG. 1.

The assessment focuses on the packaging; and the product per se (manufacture, consumption) is excluded from the assessment because it is the same for both (powder and RTD). Packaging materials manufacture, filling of product at factory, distribution, and retail of the product (ambient conditions), transport within life cycle stages, and finally the end of life management of used packaging are included in the assessment.

Five environmental impact indicators were evaluated to provide an overview of the environmental performance of packaging systems.
a) Climate change (CC) [kg CO₂-eq](100 a, IPCC, 2013). Biogenic CO₂ is assigned a characterization factor of 0. This is a midpoint indicator.
b) Freshwater consumption scarcity (FWCS) [m³-eq](AWaRE method—Available Water REmaining, v.1.0; WULCA, 2016). This is a midpoint indicator.
c) Abiotic resource depletion (ARD) [kg Sb-eq] (CIVIL 2001 method, v. 2.05; Guinee el al., 2002). This is a midpoint indicator.
d) Land use impacts on biodiversity (LUIB) [PDF·m²·year] (IMPACT World+/Land use method, v.0.05; Impact World+, 2012). This is an endpoint indicator.
e) Impacts on ecosphere/ecosystems quality (IEEQ) [PDF·m²·year] (Impact 2002+ method v. Q2.27; Jolliet et al., 2003; Humbert el al., 2012).

The packaging options considered are detailed in Table 4.

TABLE 4
Packaging formats evaluated.

| Packaging format | Sachet | Can | Bottle |
|---|---|---|---|
| Size | 20 g, 25 g | 280 g, 560 g, 840 g, | 250 ml |
| | | 1120 g | |
| Servings per format | 1 | 14, 28, 42, 56 for a 20 g | 1 |
| | | serving size | |
| | | 11, 22, 33, 44 for a 25 g | |
| | | serving size | |
| Material | Flexible multi-laminate | Steel | Rigid plastic |
| End of life management | 50% incinerated, 50% | 75% recycled, | 50% incinerated, |
| | landfilled | 12.5% incinerated, | 50% landfilled |
| | | 12.5% landfilled | |

The results for one serving size delivered via the packaging formats chosen are compared in Table 5 (for a 20 g serving size) and Table 6 (for a 25 g serving size). It shows that the single-serve packaging format for the powder product (sachet) has the lowest environmental impact relative to the cans of 4 different sizes and the 250 ml RTD bottle. Delivering the reconstituted product entails the transportation of water, whereas the associated impact is avoided by reconstitution of the product at home.

TABLE 5
Environmental performance of 3 formats for Molico product
delivery (packaging only). Impacts per serving size (20 g).

| Environmental impact | | Sachet. | Can size | Can size | Can size | Can size | Bottle, |
|---|---|---|---|---|---|---|---|
| indicator | | 20 g | 1, 280 g | 2, 560 g | 3, 840 g | 4, 1120 g | 250 ml |
| CC | [kg CO₂-cq] | 2.33E−02 | 6.12E−02 | 4.88E−02 | 4.35E−02 | 4.36E−02 | 1.51E−01 |
| ARD | [kg Sb-eq] | 1.66E−04 | 4.16E−04 | 3.30E−04 | 2.94E−04 | 2.94E−04 | 1.38E−03 |
| IEEQ | [PDF * m² *year] | 6.71E−04 | 2.19E−03 | 1.75E−03 | 1.56E−03 | 1.57E−03 | 3.67E−03 |
| LUIB | [PDF*m² *year] | 1.33E−05 | 1.17E−03 | 9.03E−04 | 8.00E−04 | 7.97E−04 | 2.63E−03 |
| FWCS | [m³-eq] | 2.48E−03 | 7.63E−03 | 6.10E−03 | 5.42E−03 | 5.42E−03 | 1.54E−02 |

TABLE 6
Environmental performance of 3 formats for Molico product
delivery (packaging only). Impacts per serving size (25 g).

| Environmental impact | | Sachet, | Can size | Can size | Can size | Can size | Bottle, |
|---|---|---|---|---|---|---|---|
| indicator | | 20 g | 1, 280 g | 2, 560 g | 3, 840 g | 4, 1120 g | 250 ml |
| CC | [kg CO₂-eq] | 2.47E−02 | 7.65E−02 | 6.10E−02 | 5.43E−02 | 5.44E−02 | 1.51E−01 |
| ARD | [kg Sb-eq] | 1.77E−04 | 5.20E−04 | 4.13E−04 | 3.67E−04 | 3.67E−04 | 1.38E−03 |
| IEEQ | [PDF * m² *year] | 7.06E−04 | 2.74E−03 | 2.19E−03 | 1.95E−03 | 1.96E−03 | 3.67E−03 |
| LUIB | [PDF*m² *year] | 3.05E−05 | 1.46E−03 | 1.13E−03 | 9.99E−04 | 9.97E−04 | 2.64E−03 |
| FWCS | [m³-eq] | 2.71E−03 | 9.54E−03 | 7.62E−03 | 6.77E−03 | 6.77E−03 | 1.54E−02 |

### Example 5: Evaluation of Performance of Various Product Scenarios

The performance of various product scenarios is evaluated and compared in Tables 7 and 8, which show the results for one indicator, climate change (CC), and one set of potential combinations of can packaging formats, respectively. Here, it is assumed that in order to deliver products for a given period (in weeks), consumers can have 3 options of receiving the product: only sachets, only bottles, or a combination of cans of different sizes. Cans are coded as follows: size 1 to 4, 1=280 g, 2=560 g, 3=840 g, 4=1120 g. Then, the overall environmental performance of each scenario (sachets, bottles, and cans) is compared.

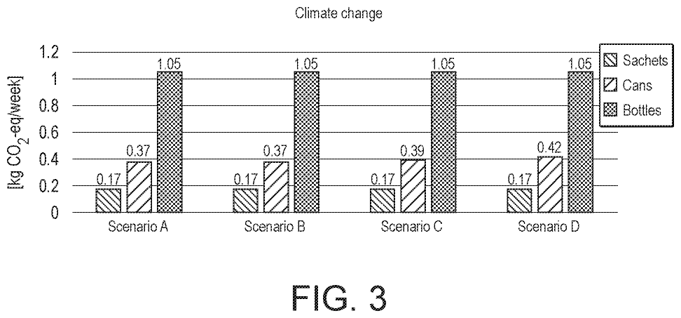

From Tables 7 and 8, delivering the product in powder using sachets or combinations of cans has a lower environmental performance than delivering reconstituted product in RTD bottles for all scenarios. Similar behavior is observed for the other 4 indicators also evaluated in the assessment. A weekly basis for comparison is shown in FIGS. 2 and 3, which summarize the results from Tables 7 and 8, respectively. FIG. 2 shows the results for the climate change impact for Scenarios A to D (Molico product) per week of nutrition delivered, linked to the combination of packaging formats detailed in Table 7. FIG. 3 shows the results for the climate change impact for Scenarios A to D (Molico product) per week of nutrition delivered, linked to the combination of packaging formats detailed in Table 8. When cans of bigger sizes are used (sizes 2 to 4), the overall impact of delivering the product diminishes further because there is a lower ratio of product to packaging.

TABLE 7
Climate change (CC) indicator for 4 scenarios of product combinations.

| | | | | | | | | | 1 can for each product. | | | | | | | |
|---|---|---|---|---|---|---|---|---|---|---|---|---|---|---|---|---|
| Product | Product | | | | | Period of | | | Can size for products | | | | | CC [kg CO₂-eq/period | | |
| combination | combination | | | | | nutrition | Sachet | Bottle | 1 to 5 | | | | | of nutrition] | | |
| scenario | 1 | 2 | 3 | 4 | 5 | [weeks] | [units] | [units] | P1 | P2 | P3 | P4 | P5 | Sachets | Cans | Bottles |
| A | 3 | 2 | 2 | | | 4 | 28 | 28 | 1 | 1 | 1 | | | 0.67 | 1.96 | 4.22 |
| B | 4 | 1 | | | 2 | 3 | 21 | 21 | 1 | 1 | | | 1 | 0.50 | 1.42 | 3.16 |
| C | 2 | 4 | | | 1 | 2 | 14 | 14 | 1 | 1 | | | 1 | 0.34 | 1.01 | 2.11 |
| D | | 1 | | 4 | 2 | 2 | 14 | 14 | | 1 | | 1 | 1 | 0.35 | 1.07 | 2.11 |

TABLE 8
Climate change (CC) indicator for 4 scenarios of product combinations;
alternative choice of packaging formats for cans.

| | | | | | | Period | | | 1 can for each product. | | | | | | | |
|---|---|---|---|---|---|---|---|---|---|---|---|---|---|---|---|---|
| Product | Product | | | | | of | | | Can size for products | | | | | CC [kg CO₂-eq/period | | |
| combination | combination | | | | | nutrition | Sachet | Bottle | 1 to 5 | | | | | of nutrition] | | |
| scenario | 1 | 2 | 3 | 4 | 5 | [weeks] | [units] | [units] | P1 | P2 | P3 | P4 | P5 | Sachets | Cans | Bottles |
| A | 3 | 2 | 2 | | | 11 | 77 | 77 | 3 | 2 | 2 | | | 1.86 | 4.12 | 11.59 |
| B | 4 | 1 | | | 2 | 11 | 77 | 77 | 4 | 1 | | | 2 | 1.84 | 4.10 | 11.59 |
| C | 2 | 4 | | | 1 | 11 | 77 | 77 | 2 | 4 | | | 1 | 1.87 | 4.31 | 11.59 |
| D | | 1 | | 4 | 2 | 11 | 77 | 77 | | 1 | | 4 | 2 | 1.90 | 4.58 | 11.59 |

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. A method of preventing and/or reducing a risk of a nutrient inadequacy, achieving a nutrient adequacy, meeting a nutrition need, and/or minimizing a nutrient gap in a subject in need thereof in an environmentally sustainable manner, the method comprising:
assessing a nutrition need of the subject;
providing combinations of existing food products that meet the nutrient need of the subject;
estimating a potential environmental impact value for each of the combinations of the existing food products;
selecting a combination of the existing food products with a lowest potential environmental impact value;
placing an order of the combination of the existing food products with the lowest potential environmental impact; and
providing a pre-packed article in reply to the order, the pre-packed article comprising the combination of the existing food products with the lowest potential environmental impact value.

2. The method according to claim 1, wherein the assessing of the nutrition need of the subject comprises calculating a nutrient gap of the subject.

3. The method according to claim 2, wherein the nutrient gap is calculated as (a daily nutrient intake recommendation−an average nutrient intake in a cluster of subjects)/the daily nutrient intake recommendation, and nutrient intakes within the cluster are more similar to each other than to those in other clusters.

4. The method according to claim 1, wherein the assessing of the nutrition need of the subject comprises using a questionnaire including about 1-10 questions.

5. The method according to claim 4, wherein the questionnaire includes about 5-6 questions.

6. The method according to claim 1, wherein the existing food products each are selected from the group consisting of a powdered nutritional composition to be reconstituted in milk or water, a powdered nutritional composition stored in a single use capsule or pod to be reconstituted in milk or water, a nutritional formula, a cereal-based product, a drink, a bar, a nutritional supplement, a pre-mix for fortification purposes, a nutraceutical, a yogurt, a dairy-based product, a food sprinkler, a gummy, a meal replacer, a pill, a tablet, and combinations thereof.

7. The method according to claim 1, wherein the existing food products each are in a form selected from the group consisting of a powder, a ready-to-drink (RTD) product, and combinations thereof.

8. The method according to claim 1, wherein the existing food products each are in a package selected from the group consisting of can, sachet, bottle, and combinations thereof.

9. The method according to claim 1, wherein the combinations of the existing food products each comprise a combination of proportions of product variants and/or numbers of product units.

10. The method according to claim 1, wherein the combinations of the existing food products each provide nutrients comprising at least one of carbohydrates, proteins, fibers, vitamins, fats, prebiotics, probiotics, vitamins, and minerals.

11. The method according to claim 1, wherein the estimating of the potential environmental impact value comprises performing a screening life cycle assessment of a factor selected from the group consisting of packaging material manufacture, filling at factory, distribution, retail, transport within life cycle stages, the end of life management of used packaging, and combinations thereof.

12. The method according to claim 11, wherein the factor does not include manufacture of the product and consumption of the existing products at home/on the go.

13. The method according to claim 1, wherein the estimating of the potential environmental impact value comprises estimating an environmental impact factor per serving size(s) delivered selected from the group consisting of climate change (CC) [kg CO2-eq], freshwater consumption scarcity (FWCS) [m³-eq], abiotic resource depletion (ARD) [kg Sb-eq], land use impacts on biodiversity (LUIB) [PDF*m²*year], and impacts on ecosphere/ecosystems quality (IEEQ) [PDF*m²*year], and combinations thereof.

14. The method according to claim 1, wherein at least one of the following steps are implemented on a computer machine:
the assessing of nutrition need of the subject;
the providing of the combinations of existing food products that meet the nutrient need of the subject;
the estimating of the potential environmental impact value for each of the combinations of the existing food products;
the selecting of the combination of the existing food products with a lowest potential environmental impact value; or
the placing of the order of the combination of the existing food products with the lowest potential environmental impact.

15. A computer system comprising:
at least one processor; and
a data storage device in communication with the at least one processor, wherein the data storage device comprises instructions that, when executed by the at least one processor, cause the at least one processor to:
assess a nutrition need of the subject;
provide combinations of existing food products that meet the nutrient need of the subject;
estimate a potential environmental impact value for each of the combinations of the existing food products;
select a combination of the existing food products with a lowest potential environmental impact value; and
place an order of the combination of the existing food products with the lowest potential environmental impact.

16. A computer readable medium comprising instructions which, when executed by a computer, cause the computer to
assess a nutrition need of the subject;
provide combinations of existing food products that meet the nutrient need of the subject;
estimate a potential environmental impact value for each of the combinations of the existing food products;
select a combination of the existing food products with a lowest potential environmental impact value; and
place an order of the combination of the existing food products with the lowest potential environmental impact.
